(19) United States
(12) Patent Application Publication
(10) Pub. No.: US 2022/0202372 A1
(43) Pub. Date: Jun. 30, 2022

(54) BIOLOGICAL-DATA PROCESSING APPARATUS, BIOLOGICAL-DATA MEASUREMENT SYSTEM, BIOLOGICAL-DATA PROCESSING METHOD, AND RECORDING MEDIUM

(71) Applicants: Hideaki Yamagata, Kanagawa (JP); Yuki Miyano, Tokyo (JP); Fumikazu Hoshi, Ishikawa (JP); Yohei Yamane, Tottori (JP)

(72) Inventors: Hideaki Yamagata, Kanagawa (JP); Yuki Miyano, Tokyo (JP); Fumikazu Hoshi, Ishikawa (JP); Yohei Yamane, Tottori (JP)

(73) Assignee: Ricoh Company, Ltd., Tokyo (JP)

(21) Appl. No.: 17/645,561

(22) Filed: Dec. 22, 2021

(30) Foreign Application Priority Data
Dec. 24, 2020 (JP) 2020-214932

(51) Int. Cl. A61B 5/00; A61B 5/248

(52) U.S. Cl. CPC A61B 5/7221; A61B 5/743; A61B 5/248

(57) ABSTRACT

A biological-data processing apparatus includes a processor; and a memory storing instructions that cause the processor to execute performing an addition-averaging process on biological data; performing a biological data process based on the biological data to obtain a process result, by using addition-average data, obtained as a result of the addition-averaging process, and a parameter that is predetermined; storing, in a storage, an addition count used to obtain the addition-average data, the addition-average data, and the parameter in association with each other; and changing a second parameter to a first parameter, in response to determining that the process result is valid when the biological data process is performed by using a first addition count that is specified and the first parameter corresponding to the first addition count, the second parameter corresponding to a second addition count that is different from the first addition count.

### CROSS-REFERENCE TO RELATED APPLICATION

The present application is based on and claims priority under 35 U.S.C. § 119 to Japanese Patent Application No. 2020-214932, filed on Dec. 24, 2020, the contents of which are incorporated herein by reference in their entirety.

### BACKGROUND OF THE INVENTION

### 1. Field of the Invention

The present invention relates to a biological-data processing apparatus, a biological-data measurement system, a biological-data processing method, and a recording medium.

### 2. Description of the Related Art

Conventionally, there is known a biological-data measurement system for measuring biological data such as magnetic field data of a living body generated in response to a stimulus such as an electrical stimulus.

Further, there is disclosed a configuration in which an addition-averaging process is performed on the biological data in order to reduce noise included in weak biological data, when performing a process based on the biological data, such as process for estimating the intensity of an activity current inside a living body (see, for example, Patent Document 1).
Patent Document 1: Japanese Patent No. 3563624

### SUMMARY OF THE INVENTION

According to one aspect of the present invention, there is provided a biological-data processing apparatus including a processor; and a memory that includes instructions, which when executed, cause the processor to execute performing an addition-averaging process on biological data; performing a biological data process based on the biological data to obtain a process result, the biological data process performed by using addition-average data, obtained as a result of the addition-averaging process, and a parameter that is predetermined; storing, in a storage, an addition count used to obtain the addition-average data, the addition-average data, and the parameter in association with each other; and changing a second parameter to a first parameter, in response to determining that the process result is valid when the biological data process is performed by using a first addition count that is specified and the first parameter corresponding to the first addition count, the second parameter corresponding to a second addition count that is different from the first addition count.

### BRIEF DESCRIPTION OF THE DRAWINGS

FIG. 1 is a diagram illustrating the overall configuration example of a biological-data measurement system according to an embodiment of the present invention;

FIG. 2 is a diagram for explaining an example configuration of a measurement apparatus according to an embodiment of the present invention;

FIG. 3 is a block diagram of an exemplary hardware configuration of a computer according to an embodiment of the present invention;

FIG. 4 is a block diagram illustrating an exemplary functional configuration of a measurement WS according to a first embodiment of the present invention;

FIG. 5 is a block diagram illustrating a functional configuration of a data storage server according to the first embodiment of the present invention;

FIG. 6 is a block diagram illustrating an example of the functional configuration of an analysis WS according to the first embodiment of the present invention;

FIG. 7 is a flowchart illustrating an example of the operation of a measurement WS according to the first embodiment of the present invention;

FIG. 8 is a flowchart of an example of an operation of the analysis WS according to the first embodiment of the present invention;

FIG. 9 is a diagram illustrating an example of an addition-average data list according to the first embodiment of the present invention;

FIG. 10 is a diagram illustrating another example of an addition-average data list according to the first embodiment of the present invention;

FIG. 11 is a diagram illustrating an example of a screen for specifying a predetermined count and a total addition count according to the first embodiment of the present invention;

FIG. 12 is a diagram illustrating an example of a measurement screen and an operation screen according to the first embodiment of the present invention;

FIG. 13 is a diagram illustrating an example of the display screen during estimation of an activity current according to the first embodiment of the present invention;

FIG. 14 is a diagram illustrating an example of a display screen of an inappropriate result of estimation of an activity current according to the first embodiment of the present invention;

FIG. 15 is a diagram illustrating an example of a display screen of an appropriate result of estimation of an activity current according to the first embodiment of the present invention;

FIG. 16 is a block diagram illustrating a functional configuration of a data storage server according to a second embodiment of the present invention.

### DESCRIPTION OF THE EMBODIMENTS

In the configuration disclosed in Patent Document 1, there are cases where the a parameter, used in the process based on the biological data, cannot be set within a short period of time.

A problem to be addressed by an embodiment of the present invention is to set the parameter used in a process based on biological data, within a short period of time.

Hereinafter, an embodiment for carrying out the present invention will be described with reference to the drawings. In each drawing, the same elements are denoted by the same reference numerals, and overlapping descriptions may be omitted.

The following embodiments are examples of a biological-data processing apparatus that embodies the technical idea of the present invention, and the present invention is not limited to the following embodiments. Unless otherwise specified, the shape, relative arrangement, parameter values, and the like, of the elements described below are not intended to limit the scope of the present invention, but are intended to be exemplary. Further, the size, the positional relationship, and the like, of the members illustrated in the drawings may be exaggerated for the purpose of clarification.

The biological-data processing apparatus according to an embodiment is an apparatus that performs a process based on the biological data measured by a measurement apparatus. A process based on biological data is, for example, a process to estimate the activity current of a living body.

The measurement apparatus is a measurement apparatus such as a spinal magnetometer that measures, as biological data, the magnetic field data of a magnetic field of a living body generated in response to stimulus such as electrical stimulus. A spinal magnetometer is a measurement apparatus that measures a slight magnetic field caused by spinal nerve activity and enables neural activity to be visualized without injuring the body (see, for example, Ushio, Shuta et al. “Visualization of the electrical activity of the cauda equina using a magnetospinography system in healthy subjects”, Clinical Neurophysiology, Volume 130, Issue 1, January 2019, pp. 1-11).

The activity current of a living body is a weak current that flows according to a potential difference that arises when an activity potential is generated when cells or tissue of the living body are stimulated, causing the stimulated part to have a negative potential relative to the rest of the living body.

In an embodiment, the biological data, such as the magnetic field data of the magnetic field generated from the living body, is subjected to an addition-averaging process. A process is then performed based on the biological data by using the addition-average data, which is the result of the addition-averaging process, and a predetermined parameter. The addition count (the number of times of addition) used to obtain the addition-average data, the addition-average data, and the parameter are stored in association with each other.

Further, according to the embodiment, if it is determined that the process result, obtained by using a specified first addition count and a first parameter corresponding to the first addition count, is valid, a second parameter corresponding to a second addition count different from the first addition count, is changed to the first parameter.

Here, a parameter refers to information that is used in the process based on biological data and information that is predetermined prior to measuring the biological data. For example, a parameter may be predetermined information that is determined before measuring the magnetic field and used to efficiently estimate the intensity of the activity current.

The parameter includes information such as the number of times of repeating (recursively) the magnetic field measurement or the body size of the patient (subject) that is a living body subject to biological-data measurement. Such parameters are appropriately adjusted and set by the user to improve the efficiency of the estimation process when estimating the activity current.

For example, the first parameter is a proven parameter that has been most recently used to obtain a valid estimate of the intensity of the activity current. On the other hand, the estimation of the activity current using the second addition-average data corresponding to the second addition count is performed by using the addition-average data of the magnetic field data of the body part or the like of the same patient or the same living body, and only the addition count differs from the estimation using the first parameter. Therefore, it is likely that the appropriate value of the parameter is close to the first parameter.

In the embodiment, the above-described point is taken into consideration, so that when a process based on biological data, such as a process for estimating the intensity of the activity current by using the addition-average data corresponding to the second addition count is performed, the first parameter for which the process result has been determined to be valid, is used. Accordingly, the parameter used for the process based on biological data, can be set within a short period of time.

Hereinafter, an embodiment will be described by taking as an example, a biological-data measurement system including a measurement apparatus for measuring a magnetic field of a living body and a biological-data processing apparatus for estimating an activity current of a living body from the biological data measured by the measurement apparatus. In the embodiment, an example of estimating the intensity of a current flowing through a nerve in the spinal cord in a living body by applying electrical stimulation to the living body, will be described.

The “user” described above and below is a user using the biological-data measurement system. More specifically, the user may be a technician who acquires biological data by using the biological-data measurement system, or a doctor who performs medical examination or diagnosis.

### EMBODIMENT

### Overall Configuration of a Biological-Data Measurement System 1

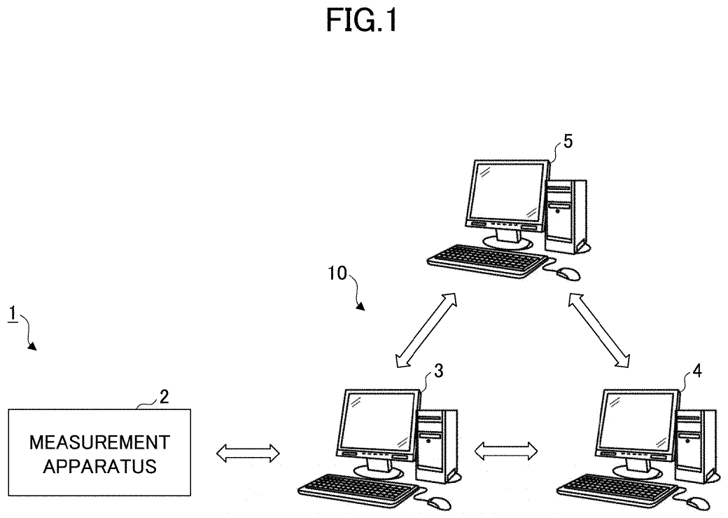

First, the overall configuration of the biological-data measurement system 1 according to the embodiment will be described with reference to FIG. 1.

FIG. 1 is a diagram illustrating an example of the overall configuration of the biological-data measurement system 1. As illustrated in FIG. 1, the biological-data measurement system 1 includes a measurement apparatus 2, a measurement WS (Work Station) 3, an analysis WS 4, and a data storage server 5. These apparatuses are communicatively connected to each other in a wired or wireless manner. Among these, the measurement WS 3, the analysis WS 4, and the data storage server 5 configure a biological-data processing apparatus 10.

The measurement apparatus 2 is a spinal magnetometer that measures magnetic field data generated in multiple parts of the living body in response to stimulus such as electrical stimulus corresponding to each of a plurality of trigger signals. Magnetic field data is an example of measurement data. The measurement apparatus 2 transmits the magnetic field data, which is a measurement result for each of the plurality of parts, to the measurement WS 3 together with a plurality of trigger signals corresponding to each of the plurality of parts.

The measurement WS 3 counts a plurality of trigger signals received from the measurement apparatus 2, acquires an addition count for each of the plurality of parts, and performs addition-averaging processing on the magnetic field data every time the addition count for each of the plurality of parts reaches a predetermined count. Then, the addition count in the addition-average data, the addition-average data that is the result of the addition-averaging process, and a predetermined parameter used for estimating the activity current of the living body, are associated with each other and transmitted to the data storage server 5.

The data storage server 5 stores, in association with each other, the addition count, the addition-average data, and the predetermined parameter received from the measurement WS 3.

The analysis WS 4 acquires the addition-average data and the parameter for each of the plurality of parts by referring to the data storage server 5, based on the addition count specified by the user, and estimates the intensity of the activity current for each of the plurality of parts by using the acquired addition-average data and parameter. The analysis WS 4 can display the estimation result, obtained by estimating the intensity of the activity current, on the display of the analysis WS 4, transmit the estimation result to the data storage server 5 to be stored, or transmit the estimation result to an external apparatus such as an external server.

The present embodiment illustrates an example of a configuration in which the biological-data processing apparatus 10 is configured by three apparatuses including the measurement WS 3, the analysis WS 4, and the data storage server 5, but the present embodiment not limited thereto. The biological-data processing apparatus 10 may be configured by a single apparatus in which the functions of the measurement WS 3, the analysis WS 4, and the data storage server 5 are integrated, or the biological-data processing apparatus 10 may be configured by four or more apparatuses over which the functions of the measurement WS 3, the analysis WS 4, and the data storage server 5 are distributed.

The biological-data measurement system 1 may include apparatuses other than the measurement WS 3, the analysis WS 4, and the data storage server 5 in a communicable manner, or may include other biological-data measurement apparatuses other than the measurement apparatus 2 in a communicable manner.

The present embodiment illustrates an example of a configuration for measuring magnetic field data of a magnetic field generated in multiple parts of the living body in response to stimulus such as electrical stimulus corresponding to each of a plurality of trigger signals, but the present embodiment not limited thereto. The configuration may be for measuring magnetic field data of a magnetic field generated in one part of the living body, in response to stimulus such as electrical stimulus according to one trigger signal.

### Configuration Example of the Measurement Apparatus 2

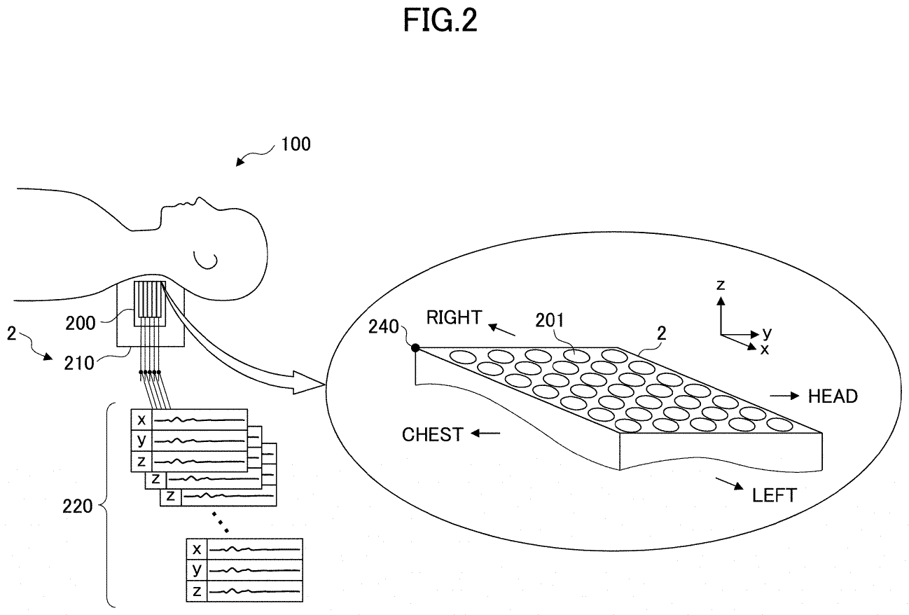

Next, the configuration of the measurement apparatus 2 will be described with reference to FIG. 2.

FIG. 2 is a diagram for explaining an example of a configuration of the measurement apparatus 2. As illustrated in FIG. 2, the measurement apparatus 2 includes a magnetic sensor array 200 and a dewar 210 that houses the magnetic sensor array 200.

The magnetic sensor array 200 is a bio-sensor including a plurality of magnetic sensors 201 arranged in an array and positioned behind the neck of a subject 100. Here, the subject 100 is an example of a “living body.”

Each of the plurality of magnetic sensors 201 measures the magnetic field of a living body in each direction of an x axis, a y axis, and a z axis illustrated by arrows in FIG. 2 and outputs magnetic field data. In the example of FIG. 2, the magnetic sensor array 200 includes 7×5 magnetic sensors, and the magnetic field data measured by each of the plurality of magnetic sensors 201 is output to the biological-data processing apparatus 10. The position where the magnetic sensor array 200 is installed with respect to the subject 100 is adjusted in advance using a marker coil or the like.

The interior of the dewar 210 is filled with liquid helium and is cooled to allow the magnetic sensor array 200 to operate at extremely low temperatures.

In an embodiment, the position of a point 240 on the magnetic sensor array 200 is the origin of the x axis, the y axis, and the z axis. By using the position of the point 240 on the magnetic sensor array 200 as the origin of the x axis, the y axis, and the z axis, the relative positional relationships between the plurality of magnetic sensors 201 in the magnetic sensor array 200 can all be represented by the x, y, and z coordinates.

Further, a known technique described in Japanese Unexamined Patent Application Publication No. 2018-089104 and the like can be applied to the method of measuring the magnetic field by the measurement apparatus 2, and, therefore, detailed descriptions thereof will be omitted here.

Further, FIG. 2 illustrates an example where the magnetic sensor array 200 is placed behind the neck of the subject 100, but in the following description, the magnetic sensor array 200 is placed behind the waist of the subject 100 to measure magnetic field data inside the living body near the waist to estimate the intensity of the current flowing through the nerves in the spinal cord. However, the estimation target is not limited to the intensity of the current flowing through the nerves in the spinal cord. For example, the intensity of current flowing through peripheral nerves in limbs such as arms and feet can be estimated.

### Example of Hardware Configuration of Computer

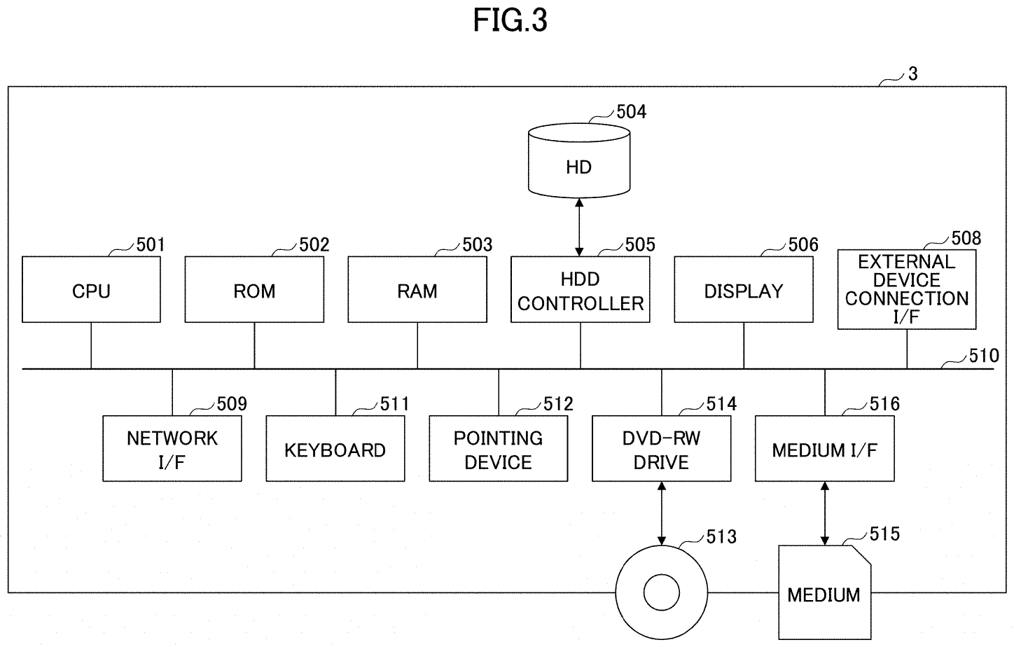

The measurement WS 3, the analysis WS 4, and the data storage server 5 according to the present embodiment can be respectively constructed by a computer. Referring to FIG. 3, the hardware configuration of the computer will be described.

FIG. 3 is a block diagram illustrating an example of a hardware configuration of a computer. FIG. 3 illustrates the hardware configuration of the computer constructing the measurement WS 3. However, the hardware configuration of the computer constructing the analysis WS 4 and the data storage server 5 is the same as that of FIG. 3.

As illustrated in FIG. 3, the measurement WS 3 includes a central processing unit (CPU) 501, a read-only memory (ROM) 502, a random access memory (RAM) 503, a Hard Disk (HD) 504, a Hard Disk Drive (HDD) controller 505, a display 506, an external device connection Interface (I/F) 508, and a network I/F 509.

Further, the measurement WS 3 includes a data bus 510, a keyboard 511, a pointing device 512, a Digital Versatile Disc Rewritable (DVD-RW) drive 514, and a medium I/F 516.

Among these, the CPU 501 controls the operation of the entire measurement WS 3. The ROM 502 stores a program used to drive the CPU 501, such as an Initial Program Loader (IPL).

The RAM 503 is used as the work area of the CPU 501. The HD 504 stores various kinds of data such as a program. The HDD controller 505 controls the reading or writing of various kinds of data from or to the HD 504 according to the control of the CPU 501.

The display 506 displays various kinds of information such as cursors, menus, windows, characters, images, or the like. The external device connection I/F 508 is an interface for connecting various external devices. In this case, the external device may be, for example, a Universal Serial Bus (USB) memory, a printer, or the like.

The network I/F 509 is an interface for performing data communication using a network. The data bus 510 is an address bus, a data bus, or the like for electrically connecting elements such as the CPU 501.

The keyboard 511 is a type of input means including a plurality of keys for input of characters, numbers, various instructions, and the like. The pointing device 512 is a type of input means for selecting and executing various instructions, selecting a processing target, moving a cursor, and the like.

The DVD-RW drive 514 controls the reading or writing of various kinds of data from or to a DVD-RW 513 that is an example of a removable recording medium. The recording medium is not limited to a DVD-RW, but may be a Digital Versatile Disc Recordable (DVD-R), etc. The medium I/F 516 controls the reading or writing (storage) of data from or to a recording medium 515, such as a flash memory.

### First Embodiment

### Example of Functional Configuration of the Biological-Data Processing Apparatus 10

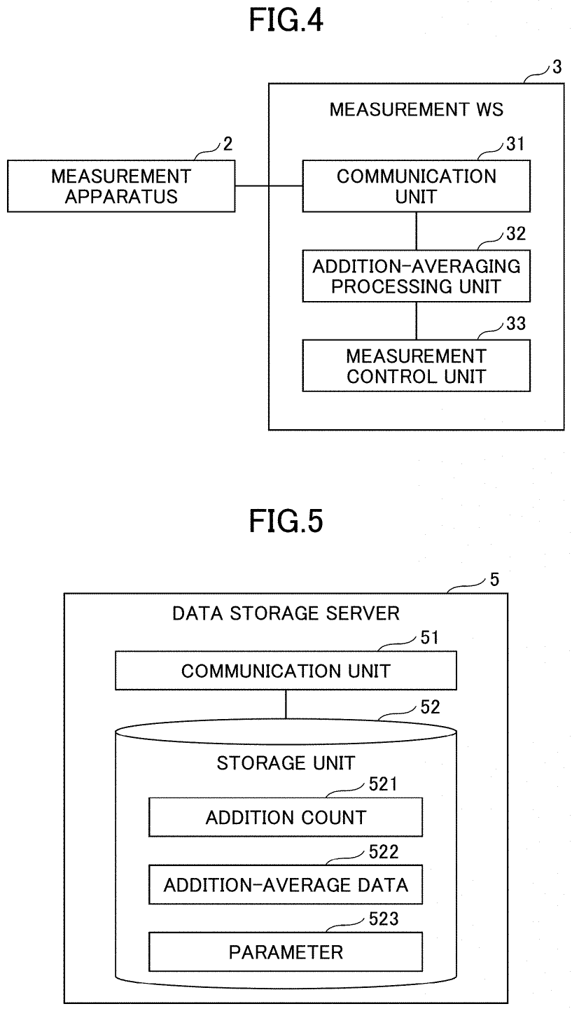

Next, the functional configuration of the measurement WS 3, the analysis WS 4, and the data storage server 5 configuring the biological-data processing apparatus 10 will be described with reference to FIGS. 4 to 6.

### Example of Functional Configuration of the Measurement WS 3

First, FIG. 4 is a block diagram illustrating an example of a functional configuration of the measurement WS 3. As illustrated in FIG. 4, the measurement WS 3 includes a communication unit 31, an addition-averaging processing unit 32, and a measurement control unit 33.

Each of these units is a function or functioning means implemented by one of the elements illustrated in FIG. 3 operating in response to an instruction from the CPU 501 according to a program loaded into the RAM 503 from the ROM 502. Although FIG. 4 illustrates the main configuration of the measurement WS 3, the measurement WS 3 may have other configurations. For example, the measurement WS 3 may include a display unit for displaying waveform data representing magnetic field data received from the measurement apparatus 2.

The communication unit 31 transmits and receives data and signals to and from the measurement apparatus 2, the analysis WS 4, and the data storage server 5.

The addition-averaging processing unit 32 acquires information of a predetermined count and the total addition count input by a user by using the keyboard 511 (see FIG. 3) or the like. The total addition count is the total number of times that the addition-averaging processing unit 32 adds magnetic field data. The addition-averaging processing unit 32 may acquire information of the predetermined count and the total addition count stored in advance in the HD 504 or the like by referring to the HD 504 or the like.

The addition-averaging processing unit 32 receives a plurality of trigger signals from the measurement apparatus 2 via the communication unit 31. The addition-averaging processing unit 32 receives, via the communication unit 31, the magnetic field data measured by the measurement apparatus 2 at predetermined intervals from the time when the magnetic field measurement is started, for each of a plurality of parts corresponding to each of the plurality of trigger signals, and performs addition processing.

The addition-averaging processing unit 32 counts the received plurality of trigger signals and acquires the addition count for each of the plurality of parts, and performs addition-averaging processing with respect to the magnetic field data for each of the plurality of parts, every time the addition count for each of the plurality of parts reaches a predetermined count.

The addition-averaging process is a process of calculating an average value by dividing, by the addition count, a value obtained by sequentially performing an addition process of adding the magnetic field data measured by the measurement apparatus 2. The addition-averaging processing unit 32 associates the addition-average data that is the result obtained by the addition-averaging process, the information on the addition count in the addition-average data, and the parameter used for estimating the activity current of the living body with each other, and transmits this associated information to the data storage server 5 via the communication unit 31.

The measurement control unit 33 receives, via the communication unit 31, an instruction based on the estimation result of estimating the intensity of the activity current obtained by an estimating unit (described later) of the analysis WS 4, and can cause the measurement apparatus 2 to discontinue the measurement based on the estimation result. The measurement control unit 33 can receive an instruction to discontinue the measurement from the analysis WS 4, as interruption data at any time when the instruction is given.

### Example of Functional Configuration of the Data Storage Server 5

Next, FIG. 5 is a block diagram illustrating an example of a functional configuration of the data storage server 5. As illustrated in FIG. 5, the data storage server 5 includes a communication unit 51 and a storage unit 52.

Each of these units is a function or functioning means implemented by one of the elements illustrated in FIG. 3 operating in response to an instruction from the CPU 501 according to a program loaded into the RAM 503 from the ROM 502. Although FIG. 5 illustrates the main configuration of the data storage server 5, the data storage server 5 may have other configurations.

The communication unit 51 transmits and receives data and signals to and from the measurement WS 3 and the analysis WS 4.

The storage unit 52 stores addition-average data 522 received from the measurement WS 3 via the communication unit 51, an addition count 521 used for the addition-averaging processing, and a parameter 523 used for the measurement of the activity current of the living body, in association with each other. The addition-average data 522 is a generic term of a plurality of pieces of addition-average data stored in the storage unit 52. The addition count 521 is a generic term of information of a plurality of addition counts stored in the storage unit 52, and the parameter 523 is a generic term of information of a plurality of parameters stored in the storage unit 52.

For example, the storage section 52 stores the information of a plurality of addition counts including a first addition count and a second addition count as the addition count 521. The information of a plurality of pieces of addition-average data including first addition-average data and second addition-average data is stored as the addition-average data 522, and information of a plurality of parameters including a first parameter and a second parameter is stored as the parameter 523.

Further, the storage unit 52 stores a first addition count, first addition-average data, and a first parameter in association with each other, and stores a second addition count, second addition-average data, and a second parameter in association with each other.

### Example of Functional Configuration of the Analysis WS 4

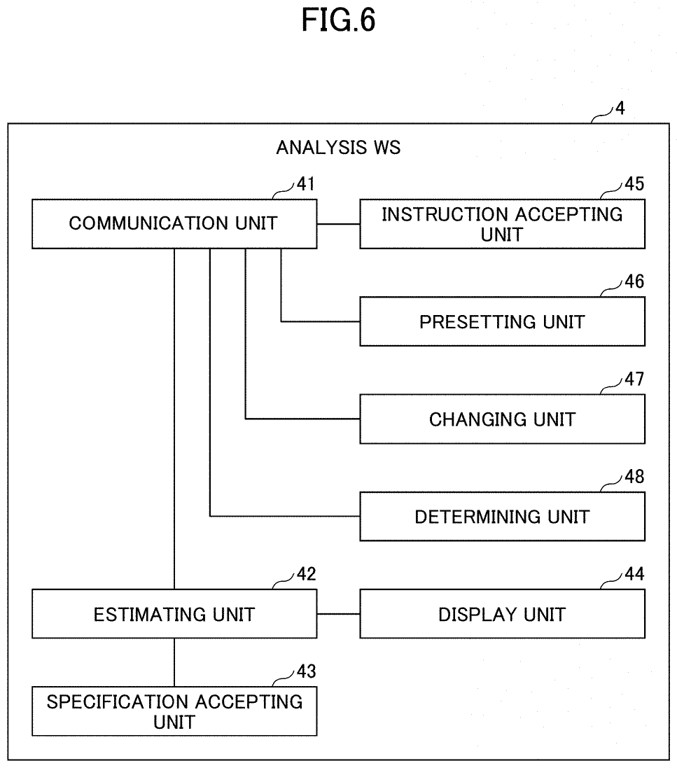

Next, FIG. 6 is a block diagram illustrating an example of a functional configuration of the analysis WS 4. As illustrated in FIG. 6, the analysis WS 4 includes a communication unit 41, an estimating unit 42, a specification accepting unit 43, a display unit 44, an instruction accepting unit 45, a presetting unit 46, a changing unit 47, and a determining unit 48.

Each of these units is a function or functioning means implemented by one of the elements illustrated in FIG. 3 operating in response to an instruction from the CPU 501 according to a program loaded into the RAM 503 from the ROM 502. Although FIG. 6 illustrates the main configuration of the analysis WS 4, the analysis WS 4 may have other configurations.

The communication unit 41 transmits and receives data and signals to and from the measurement WS 3 and the data storage server 5.

The specification accepting unit 43 accepts information of the addition count specified by the user using the keyboard 511 or the like. For example, the specification accepting unit 43 acquires a list of addition-average data stored in the storage unit 52 via the communication unit 41 and displays a list of acquired addition-average data on the display 506 or the like by the display unit 44. The specification accepting unit 43 can accept information of the addition count according to a result of the user's selection upon viewing the addition-average data list.

The estimating unit 42 is an example of a biological-data processing unit that performs processing based on biological data. The estimating unit 42 performs a process of estimating the intensity of the activity current of the living body based on the magnetic field data measured by the measurement apparatus 2.

Specifically, the estimating unit 42 acquires the addition-average data for each of a plurality of parts and parameters by referring to the storage unit 52 of the data storage server 5 via the communication unit 41 based on the specified addition count. The storage unit 52 stores the addition count, the addition-average data, and the parameter in association with each other for each of the plurality of parts of the living body. Therefore, by specifying the addition count, the addition-average data and the parameter corresponding to the addition count can be acquired for each of the plurality of parts.

The estimating unit 42 uses the acquired addition-average data and parameter to estimate the intensity of the activity current for each of a plurality of parts. As the estimation algorithm, it is possible to use “Array-Gain Constraint Minimum-Norm Spatial Filter With Recursively Updated Gram Matrix” (see, for example, Kumihashi, Isamu et al. “Array-Gain Constraint Minimum-Norm Spatial Filter With Recursively Updated Gram Matrix For Biomagnetic Source Imaging”, IEEE Transactions on Biomedical Engineering, Volume: 57, Issue: 6, June 2010, pp. 1358-1365) and the like.

The display unit 44 displays the estimation result of estimating the intensity of the activity current by the estimating unit 42. For example, the display unit 44 may display the estimation result on the display 506 and allow the user to view the result. The display unit 44 can receive, via the measurement WS 3, the waveform data representing the magnetic field data measured by the measurement apparatus 2, and display the waveform data as well.

Even in the middle of the measurement, the estimating unit 42 can estimate the intensity of the activity current with respect to the data (of the addition count in the middle of the measurement) that is already stored, and the user can view the estimation result obtained by the estimating unit 42 displayed by the display unit 44.

The user may view the estimation result of the intensity of the activity current displayed on the display 506 to determine whether the magnetic field data for each part has been properly measured, or whether the estimation result of the activity current is valid, and so on.

The instruction accepting unit 45 accepts an instruction based on the estimation result. Specifically, when the user views the estimation result displayed by the display unit 44 and determines that the magnetic field data is not properly measured, the user inputs an instruction to discontinue the measurement by using the keyboard 511 or the like. The instruction accepting unit 45 transmits the accepted discontinuation instruction to the measurement WS 3 via the communication unit 41. The measurement control unit 33 of the measurement WS 3 can cause the measurement apparatus 2 to discontinue the measurement in response to the instruction.

When the user views the estimation result displayed by the display unit 44 and determines that the estimation result of the activity current is valid, the user inputs an instruction to change the parameter by using the keyboard 511 or the like. The instruction accepting unit 45 can provide the accepted instruction to change the parameter to the changing unit 47 and cause the changing unit 47 to change the parameter.

Here, in estimating the activity current by the estimating unit 42, adjustment of several parameters, such as the number of times of repeating (recursively) the magnetic field measurement, is required. It is preferable to adjust these parameters while viewing the actually obtained activity current.

In many cases, the same value will be appropriate depending on the measured part and the like. Therefore, if the same parameter is used for the same measured part with only the addition count being different, it is highly likely that the activity current is appropriately obtained. There are also many cases where an appropriate parameter is guessed for each measured part.

On the other hand, when determining whether the estimation result of the activity current is valid in the middle of the magnetic field measurement, the user would like to avoid adjusting the parameter while viewing the measurement result as much as possible. If it takes time to adjust the parameter, the measurement will proceed in the meantime, such that the measurement cannot be stopped even when the measurement is inappropriate, such as when the stimulus electrode is out of position.

Further, when the addition count is insufficient in the final result, it would be too late by the time this insufficiency is recognized. If the insufficiency in the total addition count in the final result can be found out quickly, the measurement can be extended or the measurement can be performed again.

Therefore, according to the present embodiment, the presetting unit 46 can set the parameter 523 stored in the storage unit 52. Presetting means that the parameter is adjusted in advance. Accordingly, the parameter can be created before the start of the measurement, and thus the parameter can be adjusted without worrying about the progress of the measurement. The estimating unit 42 can estimate the intensity of the activity current of the living body based on the preset parameter.

Further, if it is determined that the estimation result of the intensity of the activity current estimated by using the specified first addition count and the first parameter corresponding to the first addition count is valid, the changing unit 47 performs a process of changing a second parameter corresponding to a second addition count that is different from the first addition count, to the first parameter. The changing process is, for example, a process of overwriting the information of the second parameter stored in the storage section 52, with the information of the first parameter.

For example, as will be described later, the analysis WS 4 displays the estimation result of the intensity of the activity current that is estimated by using the user-specified first addition count and the first parameter corresponding to the first addition count. When the user views the estimation result and determines that the estimation result is valid, the user makes an instruction to change the parameter via the instruction accepting unit 45.

The changing unit 47 changes, via the communication unit 41, the second parameter corresponding to the second addition count that is different from the first addition count, to the first parameter, in response to the instruction to change the parameter. Accordingly, the storage unit 52 can store the first parameter as a parameter corresponding to the second addition count.

The determining unit 48 determines whether the estimation result obtained by the estimating unit 42 corresponds to the total addition count and determines whether there is addition-average data corresponding to the total addition count. The determining unit 48 outputs a signal to give an instruction to discontinue the measurement, to the instruction accepting unit 45 in accordance with the determination results.

In addition to accepting an instruction to discontinue the measurement based on a user's determination, the instruction accepting unit 45 can also accept an instruction to discontinue the measurement given by the determining unit 48 and transmit the instruction to the measurement WS 3 via the communication unit 41.

### Example of Operation of the Biological-Data Processing Apparatus 10

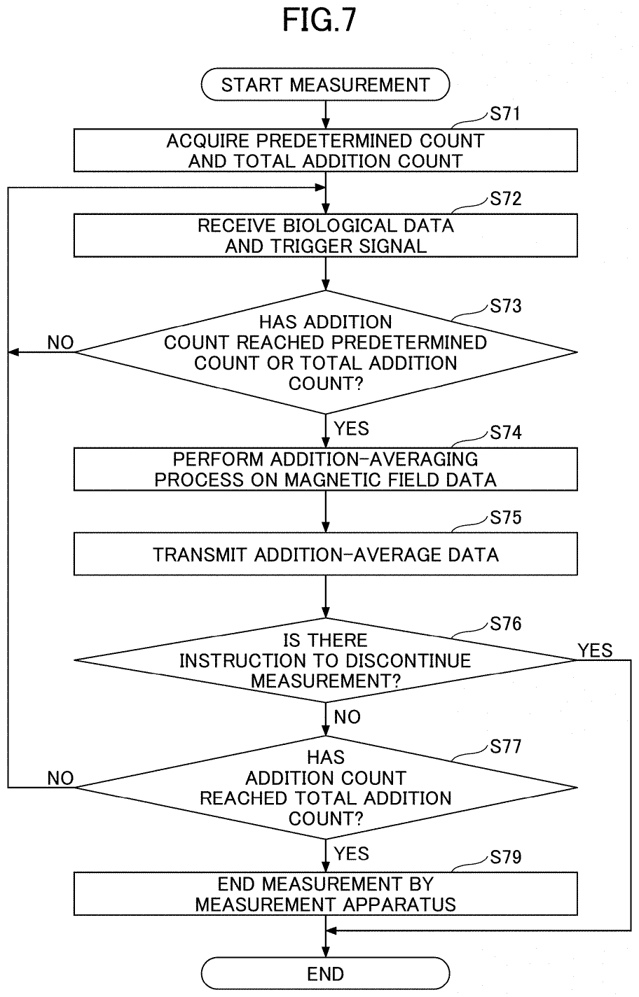

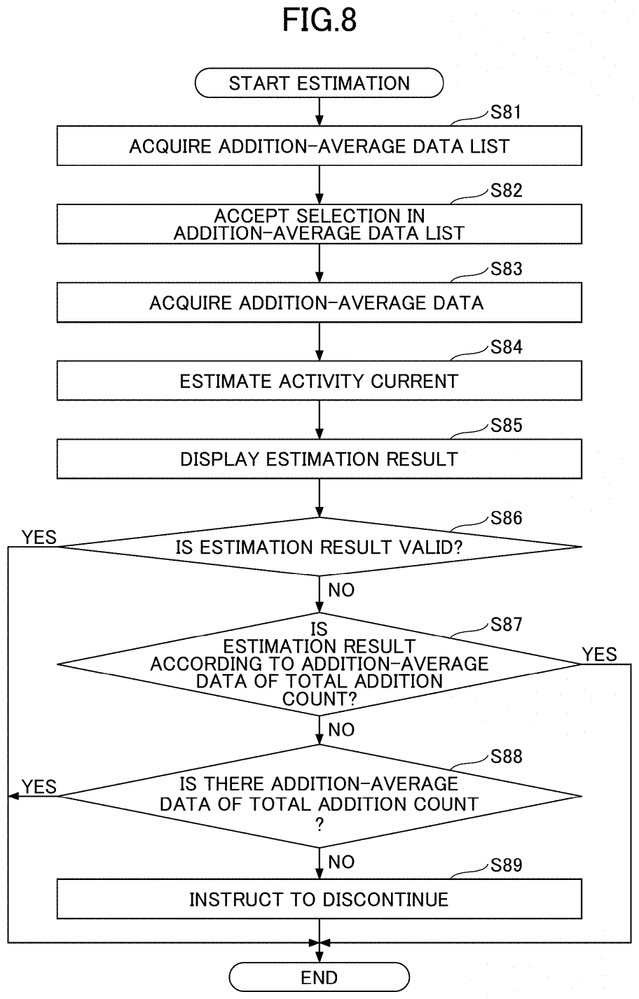

Next, the respective operations of the measurement WS 3 and the analysis WS 4 configuring the biological-data processing apparatus 10 will be described with reference to FIGS. 7 and 8.

### Example of Operation of the Measurement WS 3

First, FIG. 7 is a flowchart illustrating an example of the operation of the measurement WS 3. FIG. 7 illustrates the operation of the measurement WS 3 that is triggered at the time point when the biological-data measurement system 1 starts the measurement. At the start of the operation by the measurement WS 3, a preset parameter is stored in the storage unit 52 of the data storage server 5.

First, in step S71, the addition-averaging processing unit 32 acquires information on a predetermined count and the total addition count that the user inputs by using the keyboard 511 or the like. The addition-averaging processing unit 32 may acquire information on a predetermined count and the total addition count stored in the HD 504 or the like from the HD 504 or the like.

Subsequently, in step S72, the addition-averaging processing unit 32 receives a plurality of trigger signals and magnetic field data measured for each of the plurality of parts corresponding to each of the plurality of trigger signals, from the measurement apparatus 2 via the communication unit 31, and performs an addition process.

Subsequently, in step S73, the addition-averaging processing unit 32 counts the received plurality of trigger signals and acquires the addition count for each of the plurality of parts, and determines whether the addition count for each of the plurality of parts has reached the predetermined count.

In step S73, if it is determined that the predetermined count is reached in (YES in step S73), in step S74, the addition-averaging processing unit 32 performs addition-averaging processing on the magnetic field data for each of a plurality of parts. On the other hand, if it is determined that the predetermined count is not reached (NO in step S73), the operations from step S72 and onward are performed again.

Subsequently, in step S74, the addition-averaging processing unit 32 associates the addition-average data, which is the result of the addition-averaging processing, and information on the addition count in the addition-average data with each other, and transmits the associated information to the data storage server 5 via the communication unit 31. The data storage server 5 can store the received addition-average data and the information on the addition count in association with each other.

Subsequently, in step S76, the measurement control unit 33 determines whether an instruction to discontinue the measurement is given.

If it is determined in step S76 that an instruction to discontinue the measurement is given (YES in step S76), the operation proceeds to step S79. On the other hand, if it is determined that an instruction to discontinue the measurement is not given (NO in step S76), the operation proceeds to step S77.

The operation of step S76 is based on interrupt data from the analysis WS 4 and is performed at any timing. Accordingly, the operation of step S76 may be performed at any timing from step S71 to step S79.

Subsequently, in step S77, the measurement control unit 33 determines whether the addition count has reached the total addition count. The determination may be made by the addition-averaging processing unit 32 instead of the measurement control unit 33.

If it is determined in step S77 that the total addition count is not reached (NO in step S77), the operations from step S72 and onwards are performed again. On the other hand, in step S77, if it is determined that the total addition count is reached (YES in step S77), in step S78, the measurement control unit 33 causes the measurement apparatus 2 to end the measurement.

In this manner, the measurement WS 3 can perform the addition-averaging process and control the measurement apparatus 2 in response to an instruction to discontinue the measurement.

### Example of Operation of the Analysis WS 4

Next, FIG. 8 is a flowchart illustrating an example of an operation of the analysis WS 4. FIG. 8 illustrates the operation of the analysis WS 4 that is triggered at the time point when the user operates the analysis WS 4 to start the estimation process of estimating the activity current of the living body by using the addition-average data.

First, in step S81, the specification accepting unit 43 acquires a list of the addition-average data stored in the storage unit 52 via the communication unit 41 and the display unit 44 displays the acquired list of the addition-average data on the display 506 or the like. The user can view the displayed list of the addition-average data and select addition-average data by using the keyboard 511 or the like.

Subsequently, in step S82, the specification accepting unit 43 accepts information on the addition count based on the addition-average data selected by the user.

Subsequently, in step S83, the estimating unit 42 acquires the addition-average data for each of the plurality of parts by referring to the storage unit 52 of the data storage server 5 via the communication unit 41 based on the addition count accepted by the specification accepting unit 43.

Subsequently, in step S84, the estimating unit 42 estimates the intensity of the activity current for each of the plurality of parts by using the acquired addition-average data.

Subsequently, in step S85, the display unit 44 displays the estimation result of estimating the intensity of the activity current obtained by the estimating unit 42. For example, the display unit 44 displays the estimation result on the display 506 and allows the user to view the estimation result.

Subsequently, in step S86, the instruction accepting unit 45 accepts the determination result of whether the estimation result is valid for each part, by the user who has viewed the estimation result of the intensity of the activity current.

In step S86, if a determination result that the estimation result is valid is accepted (YES in step S86), the analysis WS 4 ends the operation. On the other hand, if a determination result that the estimation result is not valid is accepted (NO in step S86), in step S87, the determining unit 48 determines whether the estimation result is based on the addition-average data corresponding to the total addition count. Here, the determination as to whether the estimation result is valid includes a determination as to whether the estimation result is valid as data in the middle of the measurement, in a case of determining by viewing the estimated intensity of the activity current by using the data in the middle of the measurement.

If it is determined in step S87 that the estimation result is not based on the addition-average data corresponding to the total addition count (NO in step S87), in step S88, the determining unit 48 determines whether there is addition-average data corresponding to the total addition count.

In step S88, if it is determined that there is addition-average data corresponding to the total addition count (YES in step S88), the analysis WS 4 ends the operation. On the other hand, in step S88, if it is determined that there is no addition-average data corresponding to the total addition count (NO in step S88), in step S89, the instruction accepting unit 45 accepts an instruction to discontinue the measurement from the determining unit 48 and transmits the instruction to the measurement WS 3 via the communication unit 41. Thereafter, the analysis WS 4 ends the operation.

On the other hand, if it is determined in step S87 that the estimation result is based on the addition-average data corresponding to the total addition count (YES in step S87), the analysis WS 4 ends the operation.

In this manner, the analysis WS 4 can perform the estimation process of estimating the activity current of the living body and instruct the measurement WS 3 to discontinue the measurement based on the estimation result.

### Example of Operation of the Biological-Data Processing Apparatus 10

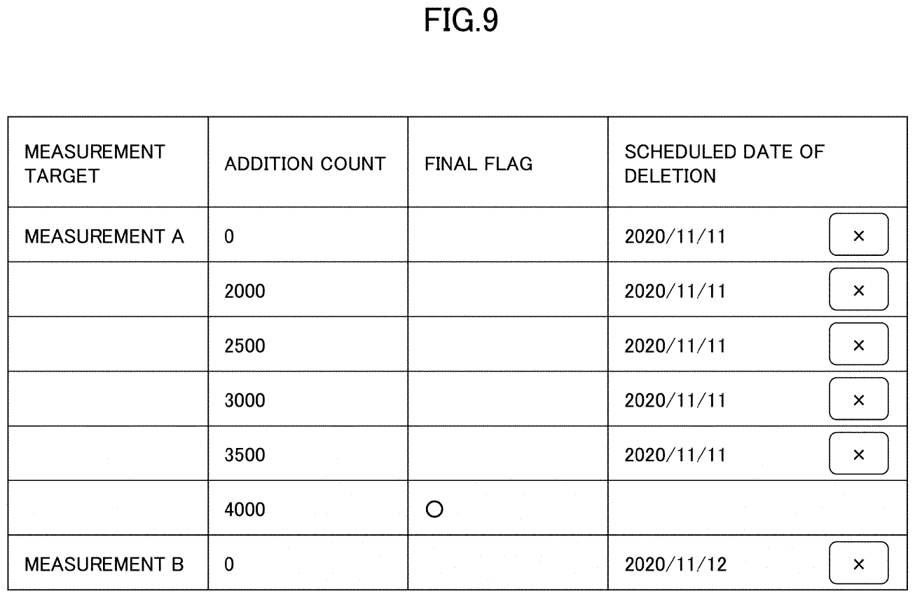

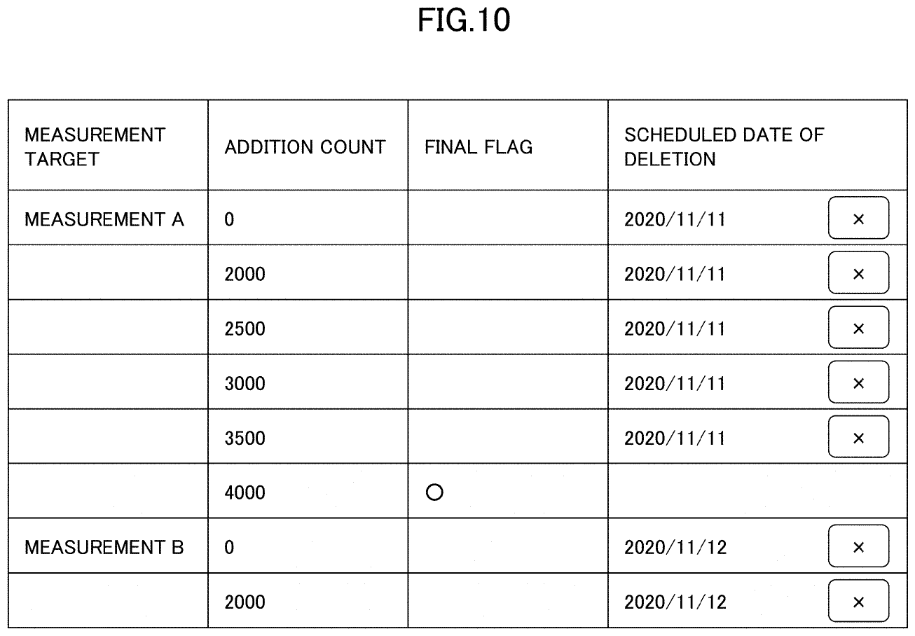

Next, an operation of the biological-data processing apparatus 10 will be described in more detail with reference to the screen examples illustrated in FIGS. 9 and 10. FIG. 9 is a diagram illustrating an example of an addition-average list according to the present embodiment, and FIG. 10 is a diagram illustrating another example of an addition-average list according to the present embodiment.

Here, FIGS. 9 and 10 illustrate the measurement target, the addition count, the final flag, and the scheduled date of deletion in each column in the stated order from the left of the table. The measurement target is information indicating a patient as a living body or a part of the living body that is the patient. The final flag is information indicating whether the addition-average data is the final addition-average data. The scheduled date of deletion indicates the scheduled date on which the addition-average data will be deleted from the addition-average list.

First, there is no addition-average data before the start of measurement, and, therefore, a typical parameter is set in advance based on the part to be measured and the like. This typical parameter is an example of a third parameter. As an example, FIG. 9 illustrates a state in which the measurement is not performed, and addition-average data, for which the addition count is 0, is created in measurement B. This processing is performed prior to step S71 in FIG. 7.

The user selects the addition-average data for which the addition count is zero in the measurement B illustrated in FIG. 9, and sets a parameter on the screen of activity current estimation. This process corresponds to the process of step S82 in FIG. 8. The presetting unit 46 stores (overwrites) the set parameter in the data for which the addition count is zero. If addition-average data for which the addition count is more than zero is generated at this time point, the presetting unit 46 stores the above-described adjusted parameter as a parameter corresponding to the generated addition-average data.

When the magnetic field data measurement progresses and the addition-average data for which the addition count is 2000 can be acquired as illustrated in FIG. 10, the specification accepting unit 43 accepts the selection of the addition-average data for which the addition count is 2000 by a user, for example. This processing corresponds to the case of storing data for which the addition count is 2000 in the processing of step S74 in FIG. 7. The estimating unit 42 estimates the intensity of the activity current by using the addition-average data for which the addition count is 2000, and the parameter stored in association with the addition-average data for which the addition count is zero that is previously stored in the same measurement. The display unit 44 displays the estimation result.

The user views the estimation result, and the instruction accepting unit 45 accepts a determination result by the user as to whether the intensity of the activity current for each part is valid. This processing corresponds to the processing of step S86 in FIG. 8.

When the estimation result of the activity current according to the parameter in the addition-average data for which the addition count is 0, is not valid, and the parameter is adjusted, the changing unit 47 changes the parameter (the second parameter) associated with the addition-average data for which the addition count is 2000, to the adjusted parameter (the first parameter). This processing corresponds the case where the parameter is adjusted in the processing of step S86 in FIG. 8, and a valid result cannot be obtained, so the process proceeds to step S87.

When the addition-average data for which the addition count is 2000 or more is generated at this time point, the changing unit 47 stores the above-described adjusted parameter as a parameter corresponding to the generated addition-average data.

In this way, when determining whether the intensity of the activity current is valid for each part in the middle of the measurement, by sequentially carrying over the parameter for estimating the activity current, the number of changes can be reduced. Further, with respect to the addition-average data that is finally obtained, by carrying over the parameter adjusted in the middle of the measurement, the activity current estimation process can be facilitated.

### Examples of Various Display Screens

Next, various display screens displayed by the biological-data measurement system 1 will be described.

### Example Screen for Specifying the Predetermined Count and the Total Addition Count

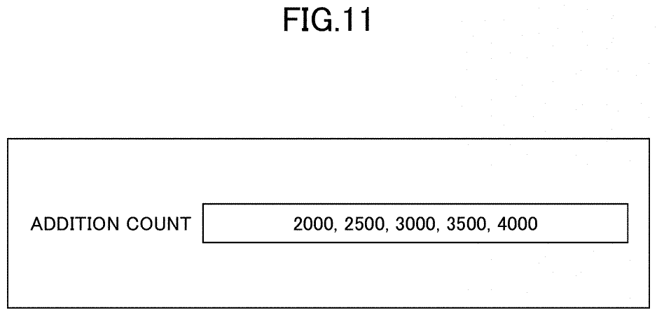

FIG. 11 is a diagram illustrating an example of a screen for specifying a predetermined count and a total addition count displayed by the measurement WS 3. The measurement WS 3 displays the screen illustrated in FIG. 11 on the display when the measurement of magnetic field data by the measurement apparatus 2 is started.

In the example illustrated in FIG. 11, 2000 times, 2500 times, 3000 times, and 3500 times each correspond to a predetermined count. Further, 4000 times corresponds to a total addition count.

In the biological-data measurement system 1, an addition-averaging process is performed every time the addition count reaches a predetermined count, and the addition-average data and the addition count in the addition-average data are stored in association with each other in the data storage server 5. Further, every time the addition count reaches the predetermined count, it is possible to estimate the intensity of the activity current for each of a plurality of parts of the living body based on the addition-average data that has undergone the addition-averaging process, and to display the estimation result, so that the user can confirm whether the measurement is being properly performed in the middle of the measurement.

### Example of Measurement Screen and Operation Screen

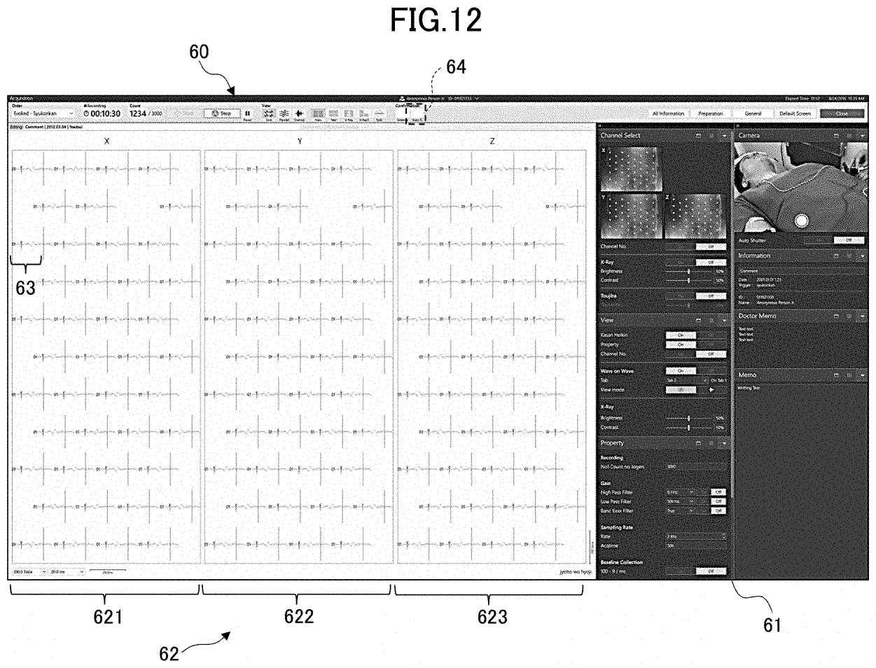

Next, the measurement screen and the operation screen will be described with reference to FIGS. 12 to 15.

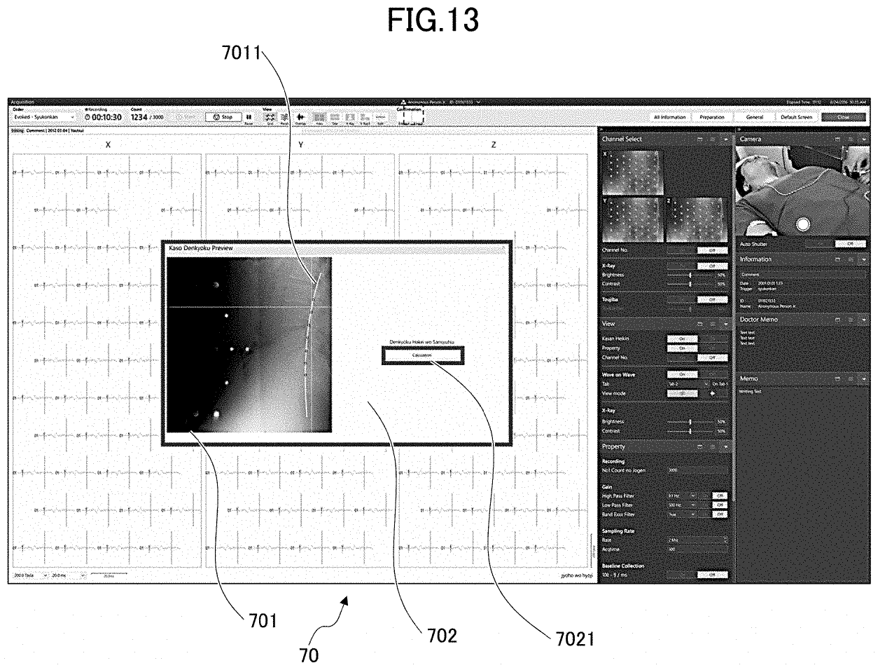

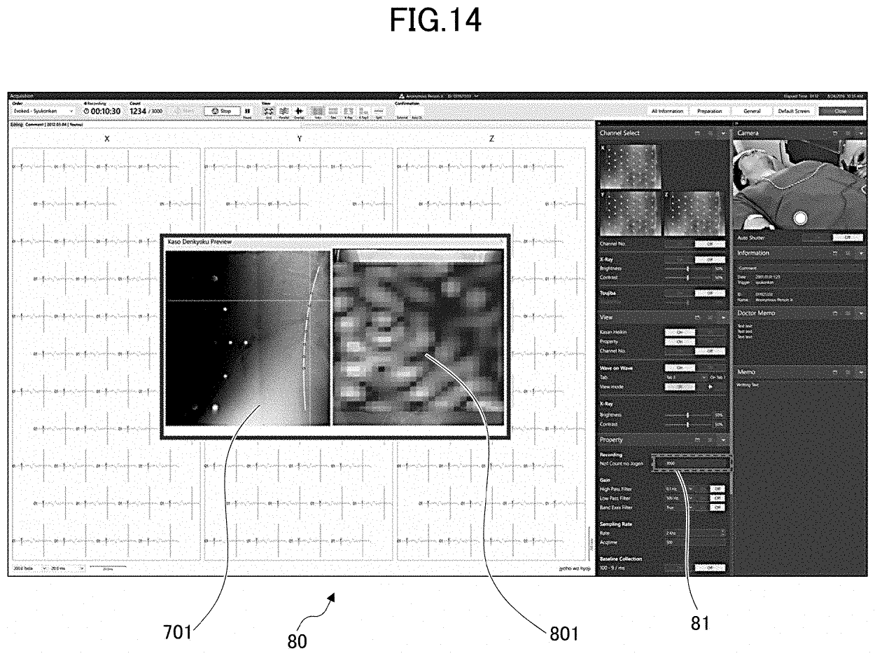

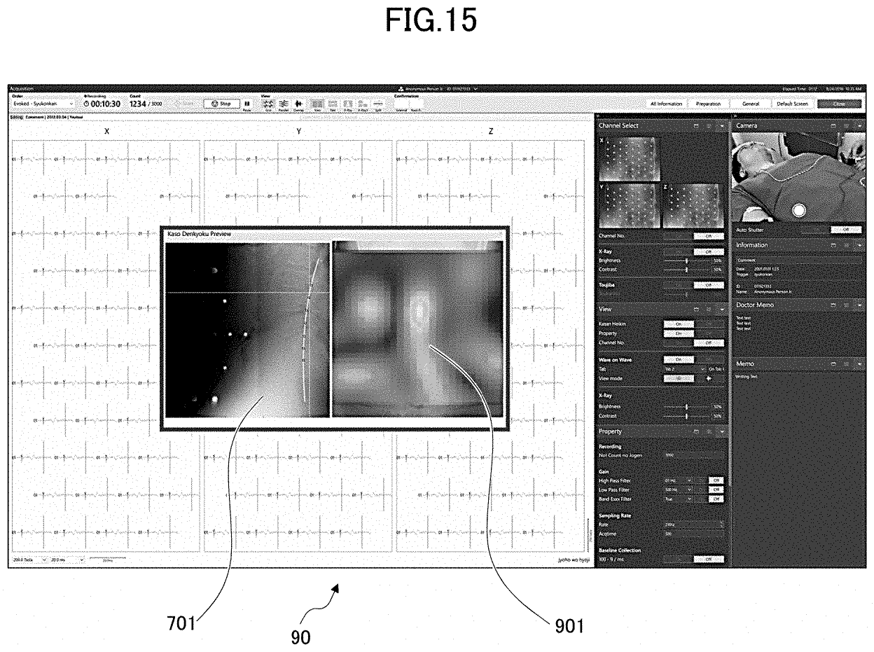

FIG. 12 is a diagram illustrating an example of a measurement screen and an operation screen displayed during measurement by the measurement apparatus 2. FIG. 13 is a diagram illustrating an example of a display screen during activity current estimation. FIG. 14 is a diagram illustrating an example of a display screen of an inappropriate estimation result of the activity current. FIG. 15 is a diagram illustrating an example of a display screen of an appropriate estimation result of the activity current.

As illustrated in FIG. 12, a measurement screen 60 includes an operation screen 61 and a measurement data screen 62. Among these, the operation screen 61 is a screen that is operated by the user to input an instruction to start the measurement, end the measurement, change the display method of the measurement data, or the like.

The measurement data screen 62 displays the magnetic field measurement data measured by the measurement apparatus 2. The measurement data screen 62 includes an x measurement data screen 621, a y measurement data screen 622, and a z measurement data screen 623.

The x measurement data screen 621 displays the magnetic field data in the x-axis direction of FIG. 2. The y measurement data screen 622 displays magnetic field data in the y-axis direction of FIG. 2, and the z measurement data screen 623 displays magnetic field data in the z-axis direction of FIG. 2.

Waveform data 63 displayed on the measurement data screen 62 displays magnetic field data obtained by one magnetic sensor included in the magnetic sensor array 200. The horizontal axis of the waveform data 63 indicates the time and the vertical axis of the waveform data 63 indicates the magnetic field intensity. The waveform data 63 displays, on a real-time basis, the magnetic field data obtained by each of a plurality of magnetic sensors included in the magnetic sensor array 200.

The number of pieces of the waveform data 63 included in each of the x measurement data screen 621, the y measurement data screen 622, and the z measurement data screen 623 corresponds to the number of the magnetic sensors included in the magnetic sensor array 200.

Here, the addition-averaging processing unit 32 performs addition-averaging processing on the waveform data 63 acquired in time series. Specifically, the addition-averaging processing unit 32 acquires the addition-average data by adding the magnetic field data per time unit in the waveform data 63 and dividing the addition result of the magnetic field data per time unit by the addition count.

Among the data included in the waveform data 63, noise-related data is generated randomly in terms of time, but the noise-related data is canceled out by performing an addition-averaging process. On the other hand, among the data included in the waveform data 63, the magnetic field data is accumulated upon being added. Thus, the addition-averaging process can amplify the magnetic field data compared to the noise-related data.

The waveform data 63 is an example of magnetic field data of the living body and is an example of biological data. Waveform data, which is generated by performing an addition-averaging process on the plurality of pieces of the waveform data 63, corresponds to the addition-average data.

In FIG. 12, a start button 64, represented by a dotted line square, is used by the user to give an instruction to start an estimation process of estimating the intensity of the activity current by the estimating unit 42. When the user presses the start button 64 by using the cursor of the pointing device 512 in FIG. 3, the instruction accepting unit 45 in the analysis WS 4 sends a request to the data storage server 5 to acquire a list of the addition-average data.

Here, after the start button 64 is pressed, a spinal cord position specifying screen 70 as illustrated in FIG. 13 may be displayed. The spinal cord position specifying screen 70 is used to specify different spinal cord positions for each subject. As illustrated in FIG. 13, the spinal cord position specifying screen 70 includes an X-ray image screen 701 and an estimation start instruction reception screen 702.

Among these, the X-ray image screen 701 displays an X-ray image taken from the side of the subject 100 (see FIG. 2). The X-ray image is an image input from an external device via the communication unit 41.

The user can specify a position for estimating the intensity of the activity current inside a living body by viewing the X-ray image screen 701 and specifying a point on the screen by using the cursor of the pointing device 512 of FIG. 3. The intensity of the activity current is estimated within the region including the specified position.

In FIG. 13, a curve 7011 included in the X-ray image screen 701 is automatically drawn so that the point specified by the user on the X-ray image screen 701 is included, and corresponds to the position of the spinal cord inside the subject 100 viewed from the side.

After specifying a point on the curve 7011, the user can press the start button 7021 in the estimation start instruction reception screen 702 by using the cursor of the pointing device 512 of FIG. 3, to start the process of estimating the intensity of the activity current at a position inside the subject 100 corresponding to the specified point.

Next, as illustrated in FIG. 14, an estimation result display screen 80 includes the X-ray image screen 701 and a distribution diagram 801 of the intensity of the activity current. The distribution diagram 801 is a diagram in which a two-dimensional distribution of the estimated intensity of the activity current is replaced by colors and displayed, based on the data measured by each magnetic sensor included in the magnetic sensor array 200.

The biological-data measurement system 1 acquires the distribution diagram 801 in time series and displays the distribution diagram 801 in time series to visualize the current flowing through the spinal cord as a video.

In the case of FIG. 14, it can be seen that in the distribution diagram 801, a strong activity current is estimated at a location where an activity current inside a living body is not supposed be present, that is, at a location outside the human body. This indicates that noise has a significant impact on the estimated intensity of the activity current. That is, FIG. 14 illustrates a case in which the estimation result of the activity current is inappropriate, because the activity current estimated based on the measurement data of the magnetic field that includes a lot of noise, because the number of pieces of measurement data used for processing by the addition-averaging processing unit 32 is insufficient.

On the other hand, in the case of FIG. 15, it can be seen that in a distribution diagram 901, a strong activity current is estimated at a location where the activity current inside the living body is supposed to be located, that is, on the spinal cord of the human body. This indicates that the effect of noise is reduced in the estimation result of the intensity of the activity current.

That is, FIG. 15 illustrates a case where the estimation result is appropriate, because the activity current is estimated based on the measurement data of the magnetic field in which noise is reduced, because the number of pieces of measurement data used for processing by the addition-averaging processing unit 32 is sufficient.

The user can view the distribution diagram illustrated in FIGS. 14 and 15 to determine whether the estimation result is appropriate. If it is determined that the estimation result is not appropriate, the user operates the start button 64 (see FIG. 12) to instruct the start of the estimation process again.

Instead of accepting the start instruction, the number of pieces of measurement data may be input as a numerical value into an edit box 81 represented by a square of a chain line in FIG. 14, and when the number of pieces of measurement data corresponding to the input number is acquired, the estimation processing by the estimating unit 42 may be started.

### Effect of the Biological-Data Processing Apparatus 10

As described above, in the embodiment, the magnetic field data is subjected to an addition-averaging process. The intensity of the activity current of the living body is estimated by using the addition-average data, which is the result of the addition-averaging process, and a predetermined parameter. The addition count of the addition-average data, the addition-average data, and the parameter are stored in association with each other.

Further, if it is determined that the estimation result of the intensity of the activity current, which is estimated by using the specified first addition count and the first parameter corresponding to the first addition count, is valid, a second parameter corresponding to the second addition count that is different from the first addition count, is changed to the first parameter.

The first parameter is a proven parameter that has been most recently used to obtain a valid estimate result of the intensity of the activity current. On the other hand, the estimation of the activity current using the second addition-average data corresponding to the second addition count is based on addition-average data of the magnetic field data of the body part or the like of the same patient or the same living body, and only the addition count differs from the estimation using the first parameter. Therefore, it is likely that the appropriate value of the parameter is close to the first parameter.

In the embodiment, when using this point to perform an estimation of the intensity of the activity current using the addition-average data corresponding to the second addition count, the first parameter by which the estimation result has been determined to be valid, is used. Accordingly, the parameter to be used to estimate the activity current, can be set within a short period of time.

In the present embodiment, the display unit 44 is provided for displaying the estimation result of the intensity of the activity current estimated by the estimating unit 42. Accordingly, the user can determine whether the estimation result of the intensity of the activity current is appropriate, and according to this determination result, the user can determine whether the second parameter is to be changed to the first parameter, or whether the measurement by the measurement apparatus 2 is to be discontinued.

Further, according to the present embodiment, the addition-averaging processing unit 32 performs the addition-averaging processing every time the addition count reaches a predetermined count. Accordingly, the storage unit 52 can store the addition count of the addition-average data and the addition-average data in association with each other.

In the present embodiment, the second addition count is less than the first addition count. Here, the noise removal effect increases as the addition count increases, and, therefore, the final result that the user wishes to obtain, corresponds to a result in the case where the addition count is large. Further, the parameter used for obtaining the result when the addition count is large, is important. Without this parameter, when data for which the addition count is large is analyzed and the parameter is adjusted, and subsequently the data for which the addition count is small is opened and adjusted, the parameter used when the addition count is large may be changed to a parameter used when the addition count is small. This is undesirable because the necessary parameter would be changed.

By limiting the second addition count to a number less than the first addition count, it is possible to prevent a situation where the parameter used when the addition count is large is changed to a parameter used when the addition count is small.

Note that when the data for which the addition count is small is later opened and analyzed, for example, it may be examined whether a sufficient result can be obtained with a smaller addition count in order to shorten the measurement time. The extent of the noise in the biological-data processing apparatus 10 is often varied, so this examination is performed to identify an appropriate addition count at an appropriate timing.

In the present embodiment, the presetting unit 46 presets the parameter stored in the storage unit 52, and the estimating unit 42 estimates the intensity of the activity current of the living body based on the preset parameter. Accordingly, the parameter can be set by using information that is known in advance, before the measurement, such as the patient's body size, and, therefore, the parameter can be set within a short period of time.

In the present embodiment, the estimating unit 42 uses the first parameter for estimating the activity current by using an addition count other than the first addition count. Accordingly, when determining whether the intensity of the activity current is valid for each part in the middle of the measurement, the parameter used for estimating the activity current is sequentially carried over, so that the number of changes can be reduced. Further, with respect to the addition-average data that is finally obtained, by carrying over the parameter adjusted in the middle of the measurement, the activity current estimation process can be facilitated.

### Second Embodiment

For example, a biological-data processing apparatus 10a can automatically delete the addition-average data other than that of the total addition count.

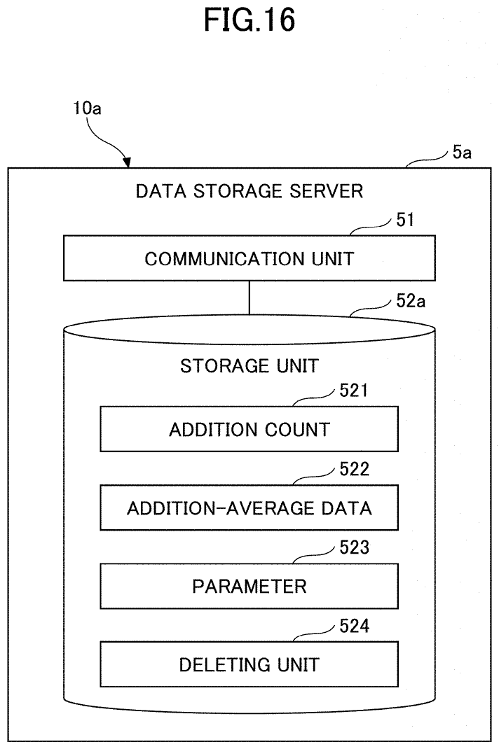

FIG. 16 is a block diagram illustrating an example of a functional configuration of a data storage server 5a in the biological-data processing apparatus 10a according to a second embodiment. As illustrated in FIG. 16, the data storage server 5a includes a storage unit 52a. The storage unit 52a includes a deleting unit 524.

When a predetermined date and time is reached, the deleting unit 524 automatically deletes the addition-average data other than that of the total addition count, for which a scheduled date of deletion is set at the predetermined date and time, among the addition-average data included in the addition-average list.

The addition-average data other than that of the total addition count is the addition-average data used to determine whether the estimation result is valid in the middle of the measurement. Therefore, as long as the addition-average data corresponding to the total addition count is stored, the addition-average data other than that of the total addition count is unnecessary after the measurement is completed.

Therefore, because the deleting unit 524 deletes such unnecessary data, the amount of the data stored in the data storage server 5a can be reduced, while also reducing the time and effort taken by the user to delete the addition-average data.

The biological-data processing apparatus, the biological-data measurement system, the biological-data processing method, and the recording medium are not limited to the specific embodiments described in the detailed description, and variations and modifications may be made without departing from the scope of the present invention.

Further, embodiments also include a biological-data processing method. For example, the biological-data processing method includes performing an addition-averaging process on biological data; performing a biological data process based on the biological data to obtain a process result, the biological data process performed by using addition-average data, obtained as a result of the addition-averaging process, and a parameter that is predetermined; storing, in a storage, an addition count used to obtain the addition-average data, the addition-average data, and the parameter in association with each other; and changing a second parameter to a first parameter, in response to determining that the process result is valid when the biological data process is performed by using a first addition count that is specified and the first parameter corresponding to the first addition count, the second parameter corresponding to a second addition count that is different from the first addition count. By such a biological-data processing method, the same effect as the biological-data processing apparatus described above can be attained.

Further, embodiments include a program. For example, a program, stored in a non-transitory computer-readable recording medium, causes a computer to execute a process. The process includes performing an addition-averaging process on biological data; performing a biological data process based on the biological data to obtain a process result, the biological data process performed by using addition-average data, obtained as a result of the addition-averaging process, and a parameter that is predetermined; storing, in a storage, an addition count used to obtain the addition-average data, the addition-average data, and the parameter in association with each other; and changing a second parameter to a first parameter, in response to determining that the process result is valid when the biological data process is performed by using a first addition count that is specified and the first parameter corresponding to the first addition count, the second parameter corresponding to a second addition count that is different from the first addition count. By such a program, the same effect as the biological-data processing apparatus described above can be attained.

The functions of each of the embodiments described above may be implemented by one or more processing circuits. As used herein, a “processing circuit” includes a processor programmed to execute each function by software such as a processor implemented in an electronic circuit; or devices such as an Application Specific Integrated Circuit (ASIC) a digital signal processor (DSP), a field programmable gate array (FPGA), and a conventional circuit module, designed to execute each function as described above.

According to one embodiment of the present invention, the parameter used in a process based on biological data can be set within a short period of time.

What is claimed is:

1. A biological-data processing apparatus comprising:
a processor; and
a memory that includes instructions, which when executed, cause the processor to execute:
performing an addition-averaging process on biological data;
performing a biological data process based on the biological data to obtain a process result, the biological data process performed by using addition-average data, obtained as a result of the addition-averaging process, and a parameter that is predetermined;
storing, in a storage, an addition count used to obtain the addition-average data, the addition-average data, and the parameter in association with each other; and
changing a second parameter to a first parameter, in response to determining that the process result is valid when the biological data process is performed by using a first addition count that is specified and the first parameter corresponding to the first addition count, the second parameter corresponding to a second addition count that is different from the first addition count.

2. The biological-data processing apparatus according to claim 1, wherein the changing includes changing a third parameter that is set in advance in an initial state, to the first parameter.

3. The biological-data processing apparatus according to claim 1, wherein
the biological data is magnetic field data of a magnetic field generated by a living body, and
the biological data process is performed by estimating an intensity of an activity current of the living body, based on the magnetic field data.

4. The biological-data processing apparatus according to claim 1, wherein the processor is further caused to execute:
displaying, on a display, the process result obtained by performing the biological data process; and
accepting an instruction based on the displayed process result.

5. The biological-data processing apparatus according to claim 1, wherein the addition-averaging process is performed every time the addition count reaches a predetermined count.

6. The biological-data processing apparatus according to claim 1, wherein the second addition count is less than the first addition count.

7. The biological-data processing apparatus according to claim 1, wherein the processor is further caused to execute:
presetting the parameter to be stored in the storage, and wherein
the biological data process based on the biological data is performed by using the parameter that is preset.

8. The biological-data processing apparatus according to claim 1, wherein the processor is further caused to execute:
automatically deleting, from the storage, the addition-average data other than the addition-average data corresponding to a total addition count.

9. The biological-data processing apparatus according to claim 1, wherein the first parameter is used in the biological data process that is performed based on the biological data using the addition count other than the first addition count.

10. A biological-data measurement system comprising:
the biological-data processing apparatus according to claim 1; and
a measurement apparatus configured to measure the biological data.

11. A biological-data processing method comprising:
performing an addition-averaging process on biological data;
performing a biological data process based on the biological data to obtain a process result, the biological data process performed by using addition-average data, obtained as a result of the addition-averaging process, and a parameter that is predetermined;
storing, in a storage, an addition count used to obtain the addition-average data, the addition-average data, and the parameter in association with each other; and
changing a second parameter to a first parameter, in response to determining that the process result is valid when the biological data process is performed by using a first addition count that is specified and the first parameter corresponding to the first addition count, the second parameter corresponding to a second addition count that is different from the first addition count.

12. A non-transitory computer-readable recording medium storing a program that causes a computer to execute a process, the process comprising:
performing an addition-averaging process on biological data;
performing a biological data process based on the biological data to obtain a process result, the biological data process performed by using addition-average data, obtained as a result of the addition-averaging process, and a parameter that is predetermined;
storing, in a storage, an addition count used to obtain the addition-average data, the addition-average data, and the parameter in association with each other; and
changing a second parameter to a first parameter, in response to determining that the process result is valid when the biological data process is performed by using a first addition count that is specified and the first parameter corresponding to the first addition count, the second parameter corresponding to a second addition count that is different from the first addition count.
